(19) United States
(12) Patent Application Publication
(10) Pub. No.: US 2012/0209059 A1
(43) Pub. Date: Aug. 16, 2012

(54) SYSTEMS AND METHODS FOR SLINGS FOR TREATING URINARY INCONTINENCE

(76) Inventor: Ian L. Goldman, Scottsdale, AZ (US)

(21) Appl. No.: 13/399,628

(22) Filed: Feb. 17, 2012

Related U.S. Application Data
(63) Continuation of application No. 12/348,840, filed on Jan. 5, 2009, now abandoned.
(60) Provisional application No. 61/018,761, filed on Jan. 3, 2008.

(51) Int. Cl. A61F 2/04

(52) U.S. Cl. 600/30

(57) ABSTRACT

Systems and methods consistent with embodiments of the present invention may delineate a system for treating urinary incontinence. The system may include a sling for contacting a portion of a patient. The system may also include a plurality of sutures coupled to the sling and a plurality of members including apertures through which the sutures may pass, wherein the plurality of members are for engagement with the sling.

### CROSS REFERENCE TO RELATED APPLICATIONS

This application is a continuation of and claims priority from U.S. patent application Ser. No. 12/348,840, filed on Jan. 5, 2009 and entitled SYSTEMS AND METHODS FOR SLINGS FOR TREATING URINARY INCONTINENCE and is further related to and claims the benefit of U.S. Provisional Patent Application No. 61/018,761, filed in the name of Dr. Ian Lee Goldman on Jan. 3, 2008, which is hereby incorporated by reference herein.

### BACKGROUND OF THE INVENTION

1. Field of the Invention

The present invention relates to systems and methods for treating urinary incontinence and more particularly, to systems and methods for slings that may be employed to treat urinary incontinence.

2. Description of the Related Art

Urinary incontinence (UI) is the involuntary loss of urine. The prevalence of male UI increases with age. There are three forms of male UI: stress incontinence, urge incontinence, and overflow incontinence. Male UI is most likely the effect of either nerve or prostate problems.

After being diagnosed with UI, men are faced with an array of treatment options, depending on the severity of UI diagnosed and the underlying cause. In the case of very mild UI, men can opt to change a few habits and do exercises. If more severe, men can be treated with medicine or an incontinence device.

Generally incontinence devices include artificial urinary sphincters, bulking materials injections, sacral nerve stimulator, bladder neck suspension, and sling procedures. During the sling procedure, a surgeon uses a strip of synthetic mesh material and places it under the urethra. The mesh acts like a hammock that compresses the urethra to prevent leaks. There are varying techniques for the sling procedure. Examples are the AdVance® and InVance® Male Sling Systems developed by American Medical Systems located in Minnetonka, Minn.

### SUMMARY OF THE INVENTION

An embodiment of the present invention may provide a system for treating urinary incontinence comprising: a sling for contacting a portion of a patient; a plurality of sutures coupled to the sling; and a plurality of members including apertures through which the sutures may pass, said plurality of members for engagement with the sling.

It is to be understood that both the foregoing general description and the following detailed description are exemplary and explanatory only and are not restrictive of the invention, as now or later claimed.

The accompanying drawings, which are incorporated in and constitute a part of this specification, illustrate several embodiments of the invention and together with the description, serve to explain the principles of the invention.

### BRIEF DESCRIPTION OF THE DRAWINGS

FIG. 1 is a plan view of an embodiment of a system in accordance with the present invention.

FIG. 2 is a side elevational view of another embodiment of a system in accordance with the present invention.

### DESCRIPTION OF THE EMBODIMENTS

Reference will now be made in detail to the present exemplary embodiments of the invention, examples of which are illustrated in the accompanying drawings.

Systems and methods consistent with embodiments of the present invention can be used to treat male patients with mild to moderate stress incontinence. Systems and methods consistent with embodiments of the present invention simplify the implantation approach of a canopy mesh sling for male urinary incontinence.

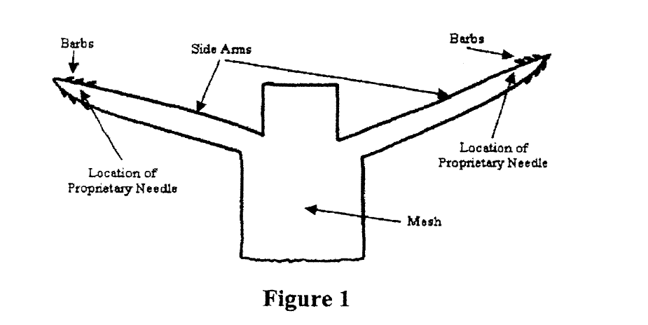

One embodiment representative of the prevent invention and shown in FIG. 1 may include a canopy mesh, side arms, and barb(s). The mesh can be made of synthetic or biological material. The mesh comprises elongated side arms. At the distal end of each side arm is multi-pronged barb(s) for future secured, bilateral attachment to the obturator internus muscles. The barb(s) can be attached to a proprietary needle. The needle drives the side arms into the obturator internus muscle, tightening and securing the canopy mesh over the bulbous urethra by a Chinese-finger-trap mechanism. One method of employing this embodiment is as follows:

1. Make a single longitudinal perineal incision with subsequent exposure of the bulbocavernous muscle.

2. Expose the bulbocavernous muscle.

3. Expose the bulbous urethra by splitting the bulbocavernous muscle in the midline.

4. Improve mobility of the bulbous urethra by dissecting the central tendon of the perineum or perineal body.

5. Cover the bulbous urethra with the canopy mesh sling which side portions are attached at the midportion of the sling and distal appendages.

6. Access the obturator internus muscle by utilizing a proprietary needle with the appropriate arc to enter the obturator space either (a) around the inferior ischial rami between the bulbous urethra and decussation of the crus of the penis or (b) between the bulbous urethra and corpus cavernosa where the latter descend.

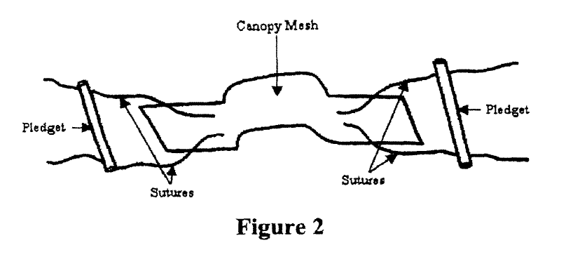

Another embodiment representative of the prevent invention and shown in FIG. 2 may include of the invention comprises a canopy mesh, pledgets, and sutures. A pledget is attached to each side of the mesh. The sutures run through each cylindrical pledget. One method of employing this embodiment is as follows:

1. Make a single longitudinal perineal incision with subsequent exposure of the bulbocavernous muscle.

2. Expose the bulbocavernous muscle.

3. Expose the bulbous urethra by splitting the bulbocavernous muscle in the midline.

4. Improve mobility of the bulbous urethra by dissecting the central tendon of the perineum or perineal body.

5. Cover the bulbous urethra with the canopy mesh sling which has not side arms.

6. Secure the edges of the mesh to the bulbous urethra with sutures, laterally via ischiocavernous muscles and inferiorly via perineal body.

7. Secure the tightening or compression of the bulbous urethra by running pledgets down toward the lateral edges of the mesh.

8. Maintain the proper tension by tying the two sutures running through each pledget.

The device and method lessen operative time, minimizes the risk of injury to structures within the obturator fossa, and provide enough compression of the bulbous urethra to maintain adequate continence in appropriately selected patients.

The above-described sling can also be used to treat urinary and fecal incontinence for both males and females.

The following patents and publications are herein incorporated by reference in their entirety: U.S. Pat. Nos. 6,911,003; 7,070,556; 2006/0287571; 2006/0235262; 2006/0252980; 2006/0195007; 2006/0195010; 2006/0195011 and 2006/0069301.

Purposes of embodiments of the present invention include developing and implementing a simpler and easier approach for the implantation of a “canopy” mesh sling for male urinary incontinence.

An embodiment of the present invention, as represented in FIG. 1, may include a single longitudinal perineal incision with subsequent exposure of the bulbocavernosus muscle following exposure of the bulbocavernosus muscle, proceed with exposure of the bulbous urethra by splitting the bulbocavernosus muscle in the midline, dissect the central tendon of the perineum or perineal body to improve mobility of the bulbous urethra. Cover the bulbous urethra with the canopy mesh whose side-arms are attached at the midportion of the sling; and whose distal appendages have multiple barbs for future secure attachment to the obturator internus muscles bilaterally. Access to obturator internus muscles is accomplished utilizing a proprietary needle with the appropriate arc to enter the obturator space around the inferior ischial rami between the bulbous urethra and decussation of the crus of the penis or between the bulbous urethra and corpus cavernosa where the latter descend. The advantages of this procedure are to lessen operative time while minimizing the risk of injury to structures within the obturator fossa and yet provide enough compression of the bulbous urethra to maintain adequate continence in appropriately selected patients.

To avoid the obturator fossa altogether and with reference to FIG. 2, another approach would be to perform the dissection as above and then place a canopy mesh without side-arms over the bulbous urethra. Secure the edges of the mesh lateral to the bulbous urethra (ischiocavernosus muscles) as well as inferiorly (attach to the perineal body) with sutures and secure the tightening or compression of the bulbous urethra by having pre-placed (along with and attached to the lateral aspects of the mesh) cylindrical pledgets with two sutures running through each so that you can run the pledgets down toward the lateral edges of the mesh creating compression and then tying the two sutures running through the pledgets to maintain the proper tension and positioning of the mesh. In this way you create the compression without possible damage to the structures within the obturator fossa by a somewhat blind placement of the side-arms.

Embodiments of the present invention may provide a canopy mesh of either synthetic or biological material with elongated side-arms composed of the same mesh material. At the distal end of each side-arm is a multi-pronged barb or multiple barbs that can be attached to proprietary needles utilized to drive the side arms into the obturator internus muscles by passing the needles between the bulbous urethra and decussating corporal bodies under the inferior ischial rami and into these muscles; thus tightening (Chinese finger-trap mechanism) and securing the canopy over the bulbous urethra. Alternative embodiments of the present invention may provide a canopy mesh with two sutures running through a cylindrical pledget attached to the lateral sides of the mesh and no side-arms.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims, as presented or hereafter filed.

What is claimed is:

1. A system for treating urinary incontinence, comprising:
a sling for contacting a portion of a patient;
a plurality of sutures coupled to the sling; and
a plurality of members including apertures through which the sutures may pass, said plurality of members for engagement with the sling.
